(19) United States
(12) Patent Application Publication
(10) Pub. No.: US 2007/0037875 A1
(43) Pub. Date: Feb. 15, 2007

(54) Genistein inhibition of transthyretin amyloidosis

(75) Inventors: Jeffery W. Kelly, La Jolla, CA (US); Nora S. Green, Mechanicsville, VA (US)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(73) Assignee: The Scripps Research Institute, La Jolla, CA (US)

(21) Appl. No.: 11/504,134

(22) Filed: Aug. 11, 2006

Related U.S. Application Data
(60) Provisional application No. 60/707,361, filed on Aug. 11, 2005.

(51) Int. Cl. A61K 31/353

(52) U.S. Cl. 514/456

(57) ABSTRACT

Genistein is an excellent transthyretin amyloidogenesis inhibitor and exhibits excellent binding selectivity in plasma. Treatment of patients having transthyretin amyloidosis with therapeutic agents containing genistein is disclosed to ameliorate the disease condition.

### TECHNICAL FIELD

The present invention relates to treatments of transthyretin amyloidosis. More particularly, the invention relates to the use of genistein as a treatment of transthyretin amyloidosis.

### BACKGROUND

Senile systemic amyloidosis (SSA) is characterized by the deposition of wild type (WT) transthyretin (TTR) amyloid fibrils in the heart and peripheral nerves (Westermark, P.; et al. Proc. Natl. Acad. Sci. USA 1990, 87, 2843-2845; McCarthy, R. E.; 3^{rd}; Kasper, E. K. Clin. Cardiol. 1998, 21, 547-552). The deposition of one of >100 different TTR variants is associated with a group of diseases collectively known as familial amyloid polyneuropathy (FAP) (Saraiva, M. J.; Costa, P. P.; Goodman, D. S. J. Clin. Invest. 1985, 76, 2171-2177; Plante-Bordeneuve, V.; Said, G. Curr. Opin. Neurol. 2000, 13, 569-573). The V30M mutation is the most common FAP variant and has been found in patients in Japan, Portugal, and Sweden. Approximately 1 million African Americans are at significant risk for congestive heart failure due to the familial amyloid cardiomyopathy (FAC) variant, V122I TTR, having high penetrance (Jacobson, D. R.; et al. N. Engl. J. Med. 1997, 336, 466-473). In addition, a subset of TTR variants has recently been shown to exhibit CNS-selective amyloidosis (CNSA).

Transthyretin (TTR) functions to transport holo-retinol binding protein and thyroxine (T4) in the blood and cerebrospinal fluid (CSF) (Nilsson, S. F.; Rask, L.; Peterson, P. A. J. Biol. Chem. 1975, 250, 8554-8563; Monaco, H. L.; Rizzi, M.; Coda, A. Science 1995, 268, 1039-1041). TTR has two identical funnel-shaped thyroxine binding sites located at the dimer-dimer interface. These thyroxine binding sites can be interconverted by 2 C₂ axes oriented perpendicular to the crystallographic two-fold axis (z-axis), FIG. 2 (Sacchettini, J. C.; Kelly, J. W. Nat. Rev. Drug Discov. 2002, 1, 267-275). Typically, less than 1% of TTR in the plasma and CSF is bound to T4, allowing these sites to be targeted with other small hydrophobic molecules to prevent amyloidogenesis (Bartalena, L.; Robbins, J. Clin. Lab. Med. 1993, 13, 583-598).

Several classes of compounds capable of inhibiting TTR fibril formation by binding to the thyroxine sites have been reported (Adamski-Werner, S. L.; et al. J. Med. Chem. 2004, 47, 355-374; Baures, P. W.; et al. Bioorg. Med. Chem. 1999, 7,1339-1347; Baures, P. W.; Peterson, S. A.; Kelly, J. W. Bioorg. Med. Chem. 1998, 6, 1389-1401; Johnson, S. M.; et al. J. Med. Chem. 2005, in press; Klabunde, T.; et al. Nat. Struct. Biol. 2000, 7, 312-321; Oza, V. B.; et al. J. Med. Chem. 2002, 45, 321-332; Peterson, S. A.; et al. Proc. Natl. Acad. Sci. USA 1998, 95, 12956-12960; Petrassi, H. M.; et al. J. Am. Chem. Soc. 2000, 122, 2178-2192; Razavi, H.; et al. Bioorg. Med. Chem. 2005, 15, 1075-1078; Razavi, H.; et al. Angew. Chem. Int. Ed. Engl. 2003, 42, 2758-2761; Wiseman, R. L.; et al. J. Am. Chem. Soc. 2005, in press). Ideally, good inhibitors should bind with high affinity, dissociate slowly, and exhibit high binding selectivity to TTR in the blood. These molecules exert their effects through kinetic stabilization mediated by preferential binding to the native state over the dissociative transition state (Hammarstrom, P.; et al. Science 2003, 299, 713-716). Kinetic stabilization of the native state is the same mechanism operating in compound heterozygotes where incorporation of T119M trans-suppressor subunits into tetramers otherwise composed of V30M subunits raises the dissociation activation barrier, thereby ameliorating disease (Hammarstrom, P.; et al. Science 2003, 299, 713-716; Hammarstrom, P.; Schneider, F.; Kelly, J. W. Science 2001, 293, 2459-2462). Similarly, small molecule kinetic stabilization of TTR is capable of ameliorating TTR amyloidosis.

Genistein (1) is an isoflavone found in various soy foods at concentrations of 1.9-229 μg/g. An additional 71-968 μg/g of genistein is present as its O-glucoside conjugate, genistin (2), which is rapidly deglycosylated by intestinal bacteria in vivo. Toxicity studies reveal that this isoflavone does not appear to cause adverse health effects, even at the relatively high concentrations employed (Okazaki, K.; et al. Arch. Toxicol. 2002, 76, 553-559; Busby, M. G.; et al. Am. J. Clin. Nutr. 2002, 75, 126-136; Bloedon, L. T.; et al. Am. J. Clin. Nutr. 2002, 76, 1126-1137).

What is needed is a highly efficacious natural product with an established safety profile in humans that binds to TTR and is efficacious for treating TTR amyloidoses.

### SUMMARY

Misfolding of transthyretin (TTR), including rate limiting tetramer dissociation and partial monomer denaturation is sufficient for TTR misassembly into amyloid and other abnormal quaternary structures associated with three amyloid diseases: senile systemic amyloidosis (SSA), familial amyloid polyneuropathy (FAP), and familial amyloid cardiomyopathy (FAC). Genistein, the major isoflavone natural product in soy, is disclosed herein to bind to one or both of the unoccupied TTR thyroid hormone binding sites. This binding is disclosed herein to have the effect of stabilizing the native tetramer more than the dissociative transition state, thereby raising the kinetic barrier for tetramer dissociation. Genistein is disclosed herein to be an excellent inhibitor of transthyretin tetramer dissociation and amyloidogenesis, reducing acid-mediated fibril-formation to less than 10% of that exhibited by TTR alone. Genistein is also disclosed herein to inhibit the amyloidogenesis of the most common FAP and FAC mutations: V30M and V122I, respectively. The binding of genistein to TTR in plasma is disclosed herein to be highly selective over all the other plasma proteins. Isothermal titration calorimetry (ITC) shows that genistein binds to TTR with negative cooperativity (K_{d1}=40 nM, K_{d2}=1.4 μM). Genistein is particularly useful as a nutraceutical to treat the orphan diseases of transthyretin amyloidosis because of its known oral bioavailability and safety data.

One aspect of the invention is directed to a method of treating a patient having or potentially having a transthyretin amyloidosis. The method comprises the step of administering to the patient a composition containing a therapeutically effective dose of genistein as an active ingredient. The genistein is administered in an amount sufficient for inhibiting acid-mediated fibril-formation of transthyretin in the plasma of said patient by at least about 90 percent during the course of said treatment. The genistein has a structure represented by the formula: 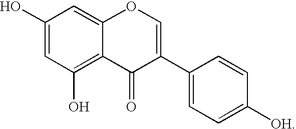
In a preferred mode, the transthyretin amyloidosis is senile systemic amyloidosis or familial amyloidosis polyneuropathy. More particularly, the familial amyloidosis polyneuropathy is characterized by V30M mutation. In another preferred mode, the transthyretin amyloidosis is familial amyloidosis cardiomyopathy. More particularly, the familial amyloidosis cardiomyopathy is characterized by a V122I mutation. In another preferred mode, the genistein is administered to said patient in an amount sufficient to raise the plasma concentration of genistein to a level of 3.6 micromolar or greater. In another preferred mode, the genistein is administered to said patient in an amount sufficient to raise the plasma concentration of genistein to a level of 7.2 micromolar or greater. In another preferred mode, the administration is repeated periodically. In another preferred mode, the genistein is administered to the patient orally.

Another aspect of the invention is directed to the use of genistein in the manufacture of a medicament for the treatment of a patient having or potentially having transthyretin amyloidosis. The medicament contains a therapeutically effective dose of genistein as an active ingredient. The genistein is administered in an amount sufficient for inhibiting acid-mediated fibril-formation of transthyretin in the plasma of said patient by at least about 90 percent during the course of said treatment. The genistein has a structure represented by the formula: 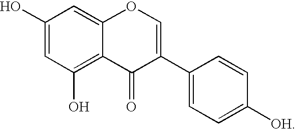
In a preferred mode, the transthyretin amyloidosis is senile systemic amyloidosis or familial amyloidosis polyneuropathy. More particularly, the familial amyloidosis polyneuropathy is characterized by V30M mutation. In another preferred mode, the transthyretin amyloidosis is familial amyloidosis cardiomyopathy. More particularly, the familial amyloidosis cardiomyopathy is characterized by a V122I mutation. In another preferred mode, the genistein is administered to said patient in an amount sufficient to raise the plasma concentration of genistein to a level of 3.6 micromolar or greater. In another preferred mode, the genistein is administered to said patient in an amount sufficient to raise the plasma concentration of genistein to a level of 7.2 micromolar or greater. In another preferred mode, the administration is repeated periodically. In another preferred mode, the genistein is administered to the patient orally.

### BRIEF DESCRIPTION OF DRAWINGS

FIG. 1 illustrates the structures of genistein (1) which is the aglycone of genistin (2), daidzein (3) and its corresponding aglycone daidzin (4) and apigenin (5) which was used for comparison with the first two aglycones.

FIG. 2 illustrates a schematic representation of the tetrameric structure of transthyretin depicting the two thyroxine binding sites.

FIG. 3 illustrates a series of three bar graphs comparing the efficacy of the different compounds in preventing fibril formation.

FIG. 4 illustrates a series of graphs showing the rate of urea-mediated tetramer dissociation (6 M) curves for (A) WT (green circles), (B) V30M, and (C) V122I TTR.

### DETAILED DESCRIPTION

The natural product genistein and several natural structurally related analogues were tested for their ability to inhibit TTR amyloid fibril formation in vitro. Genistein appears to be an exceptional inhibitor of WT TTR amyloidogenesis. Moreover, this compound exhibits highly selective binding to TTR in plasma over all other possible protein targets. Genistein also inhibits amyloidogenesis of the most common disease associated variants: V30M and V122I. The benefits of using such a nutraceutical are many, as it is possible that some patients may benefit simply from increasing their intake of soy products or adding a soy-based supplement to their diets. The wealth of toxicity information on genistein suggests that it is safe for human consumption, even at the high concentrations (Okazaki, K.; et al. Arch. Toxicol. 2002, 76, 553-559; Busby, M. G.; et al. Am. J. Clin. Nutr. 2002, 75, 126-136; Bloedon, L. T.; et al. Am. J. Clin. Nutr. 2002, 76, 1126-1137).

Discussion

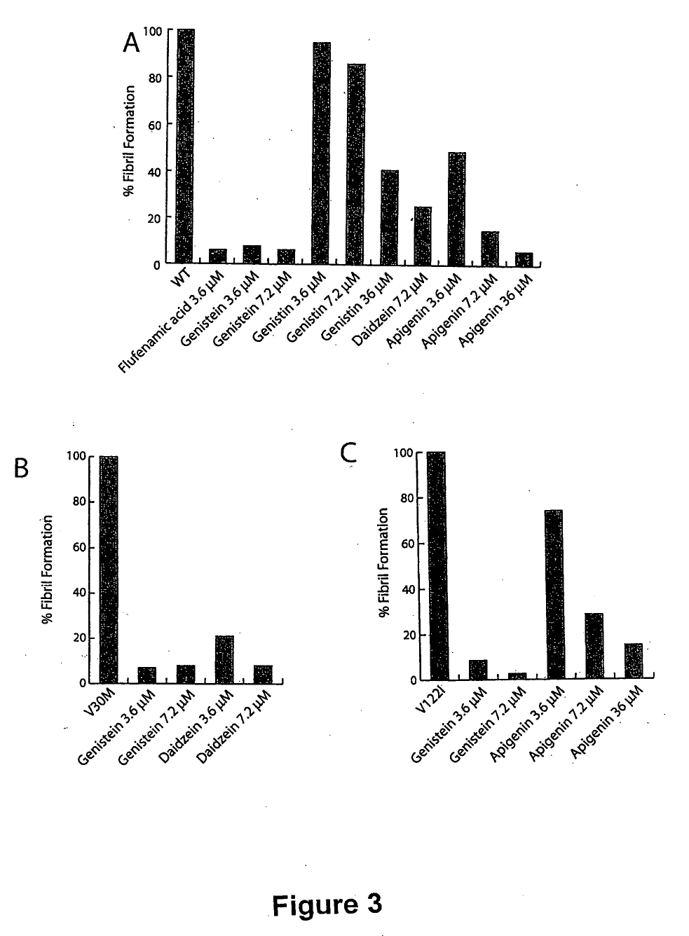

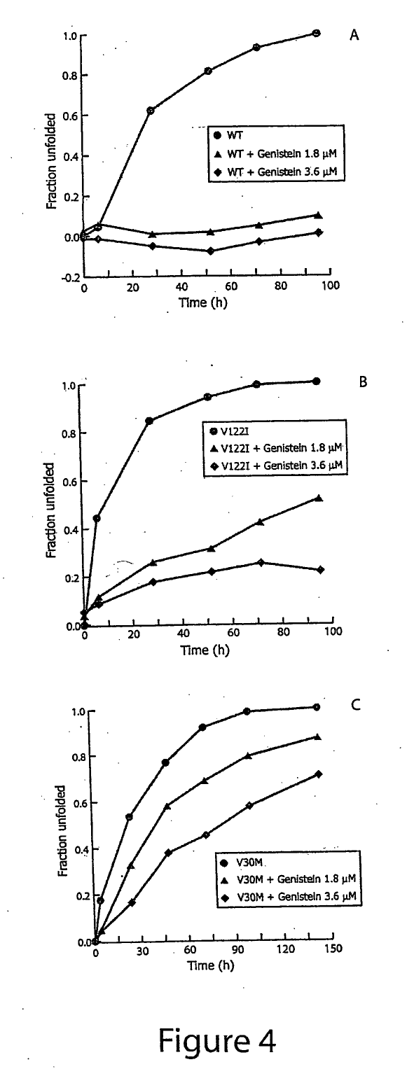

Genistein is disclosed herein to be an excellent transthyretin amyloidogenesis inhibitor. This nutraceutical substantially inhibits wild type, V30M, and V122I amyloidogenesis (FIG. 3A-C). This compound (3.6 μM or 7.2 μM) reduces fibril formation to less than 10% (3.6 μM TTR) of that exhibited by unliganded TTR. In addition, genistein dramatically slows the rate of WT and V122I TTR tetramer dissociation in urea (FIG. 4), demonstrating small-molecule mediated kinetic stabilization of the tetramer. The lesser effect seen with V30M does not necessarily imply that genistein will be inferior in treating V30M disease, as these experiments employ urea solutions that are unlikely to simulate the physiological conditions in which genistein must be efficacious, rather they are used to demonstrate kinetic stability.

Kinetic stabilization of the TTR tetramer results from selective stabilization of the native state over the dissociative transition states. Kinetic stabilization of V30M containing TTR tetramers by inclusion of T119M subunits is sufficient to ameliorate TTR amyloidosis, suggesting that genistein-mediated kinetic stabilization of TTR should be effective at preventing disease in humans. Kinetic stabilization is the most conservative strategy, as it remains unclear what species on the amyloidogenesis pathway induces toxicity.

Isothermal titration calorimetry was employed to determine the binding constants of genistein for WT TTR at pH 8.0 (25° C.). Subtraction of blanks and integration of the resulting thermogram gave a binding isotherm that fit equally well to a model of two sequential interacting binding sites with negative cooperativity (K_{d1}=40 nM, K_{d2}=1400 nM) or two identical non-interacting sites (K_{d1}=K_{d2}=845 nM). Given the strong aggregation inhibition observed at equal concentrations of genistein and TTR, it appears likely that genistein binds with negative cooperativity as K_{d2}=40 nM, K_{d2}=1400 nM affords predominantly TTR.1 in solution. The efficacy at the low concentration would not be expected if K_{d1}=K_{d2}=845 nM as unliganded TTR would be the major species.

The hydroxyl groups in the 5 and 7 positions of genistein seem to be important for aggregation inhibition. Daidzein, lacking the 5—OH, has an approximately 4-fold decrease in aggregation inhibition potency when administered at a concentration twice that of TTR (7.2 μM). Masking the hydroxyl group at the 7 position with a glucose moiety (genistin) leads to a dramatic loss of activity −41% fibril formation remaining even at very high inhibitor concentrations (36 μM genistin, 3.6 μM protein). The position of the p-hydroxy phenyl substituent also appears to be important. Moving this substructure from the 2 position of the isoflavone (genistein) to the 1 position (apigenin) results in a 2-fold decrease in WT TTR aggregation inhibition at pH 4.4.

It has been previously reported the diflunisal is efficacious as a non-steroidal anti-inflammatory drug (NSAID), for the inhibition of transthyretin amyloidogenesis (Miller, S. R.; Sekijima, Y.; Kelly, J. W. Lab. Invest 2004, 84, 545-552). While this compound shows promise in a normal human subjects oral dosing study (Sekijima, Kelly unpublished results), it may be problematic for the treatment of V122I FAC owing to compromised renal blood flow in the African American population, which suffers from a much higher incidence of kidney disease and failure (U.S. Renal Data System, National Institutes of Health, National Institutes of Diabetes and Digestive and Kidney Diseases, Bethesda, Md.). Treatment with NSAIDs will likely exacerbate this risk since they inhibit the synthesis of prostaglandins, which help to maintain blood flow to the kidneys. Genistein may be a better V22I amyloidosis inhibitor since it has not been shown to have any adverse effects on kidney function and is more active and selective than diflunisal.

A significant but not insurmountable issue is that the oral bioavailabilities of genistein and genistin are modest with in vivo plasma concentrations of genistein around 0.1 to 8 μM at a dose of 16 mg/kg of body weight (Busby, M. G.; et al. Am. J. Clin. Nutr. 2002, 75, 126-136; Bloedon, L. T.; et al. Am. J. Clin. Nutr. 2002, 76, 1126-1137; Setchell, K. D.; et al. J. Nutr. 2001, 131, 1362S-1375S). Liu and Hu's (Liu, Y.; Hu, M. Drug Metab. Dispos. 2002, 30, 370-377) study employing Caco-2 cells and perfused rat intestinal models show that genistein is efficiently absorbed into the intestine, but extensive first pass metabolism results in formation of 7—OH-glucuronic acid as the major metabolite. The permeability of genistin was approximately 5-fold lower than its corresponding aglycone. The half-life of genistein in plasma was determined to be 3.2 h for men and 3.8 h for women. These appealing pharmacokinetics suggest that a slow release formula could be useful (Busby, M. G.; et al. Am. J. Clin. Nutr. 2002, 75, 126-136; Bloedon, L. T.; et al. Am. J. Clin. Nutr. 2002, 76, 1126-1137).

Soy products, and genistein in particular, have been reported to have anti-tumor effects, through the inhibition of protein tyrosine kinase pathways leading to gene expression modification of many proteins including vascular endothelial growth factor (VEGF). These expression changes have been shown to arrest cell growth and proliferation, angiogenesis, and the cell cycle at G2/M (Ravindranath, M. H.; et al. Adv. Exp. Med. Biol. 2004, 546, 121-165). The interaction of genistein with tyrosine kinases, and their influence on numerous biological pathways poses a concern for long-term therapy. These concerns are tempered by both epidemiological data suggesting that diets high in soy have numerous positive effects, and by numerous short term high-dose studies evaluating the toxicity of genistein.

Materials and Methods

Genistein, daidzein, and apigenin were purchased from Aldrich Chemical Company. Genistin was purchased from Calbiochem and used as provided. The purity of these compounds was established by HPLC and high resolution mass spectrometry.

Protein Expression and Purification

WT, V122I, and V30M TTR were expressed and purified from E. coli as described previously (Foss, T. R.; et al. J. Mol. Biol. 2005, in press).

Stagnant Transthyretin Aggregation Assay

Stagnant aggregation assays were performed as described previously (Lashuel, H. A.; et al. Biochemistry 1999, 38, 13560-13573). A 0.495 mL sample of TTR (7.6 μM (0.4 mg/mL) in 10 mM sodium phosphate, 100 mM KCl, and 1 mM EDTA at pH 7.0) was incubated with 5 μL of isoflavone or flavone inhibitor in DMSO (0.76 mM or 1.44 mM). After 30 min, the samples were diluted with 0.5 mL of 200 mM acetate buffer (pH 4.2, final pH 4.4 for WT and V122I, pH 4.8, final pH 5.0 for V30M) containing 100 mM KCl and 1 mM EDTA. Samples were briefly vortexed and then further incubated at 37° C. for 72 h without stirring. The extent of aggregation was probed by turbidity measurements at 350 and 400 nm on an HP 8453 UV-visible spectrometer. Single-time point samples (72 h) were vortexed immediately before the measurement.

TTR Urea Denaturation Curves by Circular Dichroism

TTR (400 μL, 0.25 mg/mL; 4.5 μM tetramer) was pre-incubated with genistein at concentrations of either 4.5 or 9.0 μM for 18 h at 25° C. Urea (10M, 600 μL) in 50 mM phosphate buffer (pH 7.0) containing 100 mM KCl, 1 mM EDTA and 1 mM DTT was added to the samples immediately prior to the first measurement (1.0 mL total volume, 6.0 M urea, 0.1 mg/mL TTR (1.8 μM tetramer) final concentration). Circular dichroism spectra were recorded as a function of time up to 120 h (25° C.) using a wavelength scan from 220 to 214 nm, sampling every 0.5 nm. The signal from 218 to 215 was averaged and plotted to determine the fraction of TTR tetramer that was dissociated and unfolded at each time point.

TTR Antibody Purification and Conjugation to Sepharose

Antibodies, raised as described previously (Purkey, H. E.; Dorrell, M. I.; Kelly, J. W. Proc. Natl. Acad. Sci. USA 2001, 98, 5566-5571) were purified by passage of rabbit serum over a recombinant staphylococcal protein A column. The column was washed with 5 column volumes of 50 mM sodium phosphate pH 7.2 buffer, and the antibodies were eluted with 5 column volumes of 100 mM citrate buffer (pH 3.0). Each 5 mL elution fraction was neutralized with 1 mL of 1 M Tris-HCl buffer (pH 9.0). The fractions were then dialyzed against 100 mM sodium bicarbonate, pH 8.2. The concentrated protein was then coupled to cyanogen bromide activated Sepharose. The Sepharose gel was first washed in a filter funnel with 1400 mL of 1 mM HCl for 15 min. The coupling buffer (100 mM sodium bicarbonate, 500 mM NaCl, pH 8.3) and the antibody were added to the washed gel (5 mL coupling buffer and 35 mg antibody per gram of gel). The gel was rotated at room temperature for 1 h., followed by centrifugation at 3,000 rpm for 1 min. The gel was transferred to 100 mM Tris-HCl buffer (pH 8.0) and was rotated at room temperature for 2 h. The gel was washed with 100 mM acetate buffer (pH 4.0) containing 500 mM NaCI and 100 mM Tris-HCl buffer (pH 8.0) containing 500 mM NaCl for 2 cycles. The gel was washed twice with TSA (10 mM Tris-HCl, 140 mM NaCl, 0.025% sodium azide, pH 8.0) and stored as a 1:1 slurry in TSA.

Plasma Selectivity Binding of Genistein and Daidzein to TTR

The binding stoichiometry of genistein and daidzein to TTR in blood plasma was determined by an antibody capture/HPLC method (Purkey, H. E.; Dorrell, M. I.; Kelly, J. W. Proc. Nat. Acad. Sci. USA 2001, 98, 5566-5571). A sample of 7.5 μL of a 1.44 mM DMSO stock solution of potential inhibitor was added to a 1.5 mL Eppendorf tube containing 1.0 mL of human blood plasma. The mixture was incubated at 37° C. for 18 h. A 1:1 gel:Tris saline slurry (125 μL) of quenched sepharose was added and the resulting slurry rocked for 1 h at 4° C. The mixture was centrifuged (16,000×g) and the supernatant divided into two equal 400 μL aliquots. To each aliquot was added 200 μL of a 1:1 gel:Tris saline slurry of the anti-TTR antibody-conjugated sepharose (see above). These mixtures were rocked slowly for 20 min at 4° C., followed by centrifugation (16,000×g) and removal of the supernatant. The gel pellet was washed with 1 mL of Tris saline with 0.05% saponin (3×10 min) at 4° C., followed by 2×1 mL washes (10 min each) with Tris saline. The samples were centrifuged (16,000×g) after the final wash and 155 μL of 100 mM triethylamine (pH 11.5) was added to the resultant pellet to elute the TTR and bound small molecules from the antibody. The high pH mixture was rocked at 4° C. for 30 min and then centrifuged (16,000×g). The supernatant (145 μL) containing TTR and inhibitor was removed and analyzed by reversed phase HPLC. The resulting solution (135 μL) was injected onto a Waters 717Plus auto-sampler utilizing a Keystone 3-cm C₁₈ reverse-phase column at 100% solution A. A 20-100% linear gradient of solution B over 9 min was utilized to elute both TTR and inhibitor. Solution A is composed of 94.8% water, 5% acetonitrile, and 0.2% trifluoroacetic acid. Solution B contains 94.8% acetonitrile, 5% water, and 0.2% trifluoroacetic acid. Detection at 280 nm was accomplished with a Waters 486 tunable absorbance detector. The integrated peaks of the small molecule and TTR were compared to calibration curves prepared from known amounts of small molecule and TTR.

Isothermal Titration Calorimetry

The dissociation constants characterizing the binding of genistein to WT TTR were determined using a Microcal isothermal titration calorimeter (Microcal Inc., Northhampton, Mass.). A solution of the small molecule (final concentration 432 μM in 25 mM Tris (pH 8.0) containing 100 mM KCl, 1 mM EDTA, 10% EtOH,) was prepared and titrated into an ITC cell containing WT TTR (12 μM in 25 mM Tris (pH 8.0) containing 100 mM KCl, 1 mM EDTA, 10% EtOH). For all runs, a small preliminary injection was followed by identical injections (2.0-5.0 μL) up to a ligand:protein molar ratio of at least 4:1. The data were fit by a nonlinear least squares approach to either an identical binding sites model, or a sequential interacting binding sites model (Foss, T. R.; et al. J. Mol. Biol. 2005, in press) utilizing Microsoft Excel (Microsoft Corporation, Redmond, Wash.) with the Solver plugin.

Results

Fibril Formation Assays

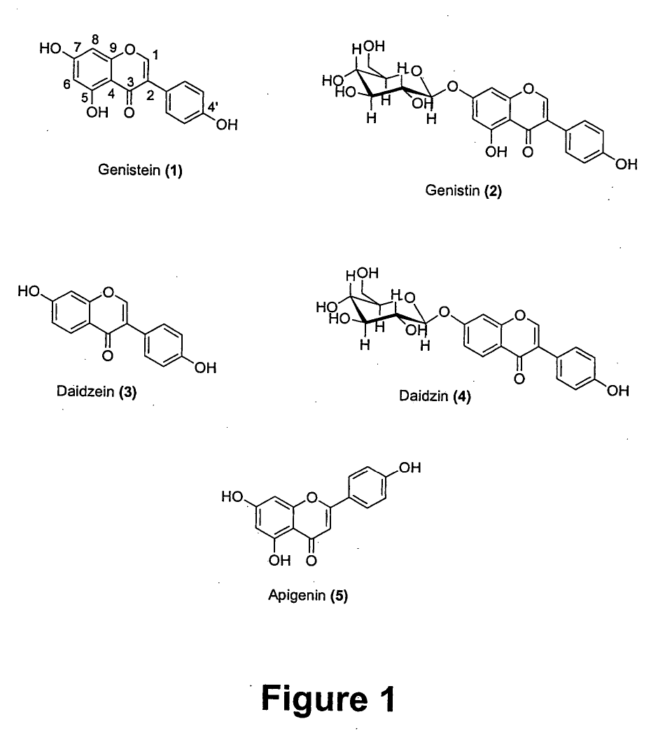

Genistein (1), genistin (2), daidzein (3), and apigenin (5, FIG. 1) were tested as potential inhibitors of WT TTR amyloidogenesis, employing a turbidity assay described previously (Lashuel, H. A.; et al. Biochemistry 1999, 38, 13560-13573). These prominent components of soy were evaluated because a soy extract exhibited activity in a screen for natural product inhibitors of TTR amyloidosis (N Green, unpublished results). Genistein was also tested for its efficacy as an amyloidogenesis inhibitor of the most common FAP and FAC mutations, V30M and V122I respectively. Aggregate formation is reported relative to WT or mutant TTR homotetramer where amount of aggregation in the absence of inhibitor is assigned to be 100%. Hence 5% aggregate formation in the presence of a given inhibitor corresponds to 95% inhibition. Genistein essentially prevented acid-mediated aggregation (2-9% fibrils) from WT, V30M, and V122I TTR (3.6 μM) at both concentrations of inhibitor tested (3.6 μM or 7.2 μM) (FIG. 3). Daidzein and apigenin were less effective inhibitors of WT aggregate formation, allowing approximately 20% and 28% aggregation respectively, when administered at a concentration twice that of TTR (7.2 μM). The glucoside genistin was a very poor inhibitor displaying 41% WT TTR aggregate formation at a concentration an order of magnitude higher (36 μM) than that of TTR.

Rate of Tetramer Dissociation as a Function of Genistein Concentration

Genistein was further tested for its ability to kinetically stabilize tetrameric TTR against urea-induced dissociation. Since dissociation of the tetramer is required for urea-induced monomer denaturation, it is possible to monitor rate-limiting tetramer dissociation by linking this process to fast monomer unfolding in a post-transition urea concentration (6 M), rendering the process irreversible. The rate and extent of tetramer dissociation at different small molecule concentrations was monitored by far-UV CD spectroscopy in 6.0 M urea. Genistein exerts its most dramatic effect on the amplitude of WT TTR tetramer dissociation (FIG. 4A). At equimolar amounts of genistein and WT TTR (1.8 μM), only 10% of the protein dissociates and unfolds after 120 h, implying that the remainder is stabilized as a consequence of small molecule binding. This compares to 33% of dissociation for V122I (FIG. 4C, triangles) and 87% V30M (FIG. 4B, triangles) dissociating under identical conditions. When the inhibitor concentration (3.6 μM) is twice that of TTR (1.8 μM), only 1% of WT TTR (FIG. 4A, diamonds), 18% of V122I (FIG. 4C, diamonds), and 70% of V30M (FIG. 4C, diamonds) dissociates over the same time period. These results are consistent with small-molecule binding imposed kinetic stabilization of the TTR tetramer.

Plasma Selectivity of Genistein and Daidzein

In order to test whether genistein and daidzein could bind selectively to TTR over all other proteins in the blood plasma, these two compounds were incubated with plasma and their binding stoichiometry to TTR determined. The two isoflavones were separately incubated with human plasma at a concentration of 10.8 μM (typical TTR concentrations in human plasma are about 5 μM). Transthyretin was captured with a resin-bound anti-TTR antibody and subjected to 3 wash steps. After high pH release of TTR and any bound small molecule from the antibody, the stoichiometry of inhibitor binding to TTR was evaluated by reverse phase HPLC. A maximum of 2.0 equivalents of inhibitor may be bound per TTR tetramer, and the possibility of wash-associated losses lowering the observed stoichiometry means these numbers should be considered a lower limit. An analysis of four separate experiments reveals a plasma selectivity for genistein of 1.45 equivalents per tetramer, implying that wash associated losses and dissociation constant of the ligand are very low. Daidzein, on the other hand, displays a binding stoichiometry of 0.75, which is a lower limit.

Determination of Genistein's Binding Constants to WT TTR

Isothermal titration calorimetry was employed to determine the dissociation constants of the binding of genistein to WT TTR at pH 8.0 (25° C.). Integration of the thermogram after subtraction of blanks yielded a binding isotherm that fit equally well to a model of two sequential interacting binding sites with negative cooperativity or two identical non-interacting sites. The fit to sequential binding sites yielded dissociation constants of K_{d1}=40±25 nM, K_{d2}=1400±170 nM. Fitting the data to identical binding sites gave K_{d1}=K_{d2}=845±45 nM with an occupancy of 1.92±0.07. Inhibition efficacy strongly suggests negatively cooperative binding (K_{d1}=40±25 nM, K_{d2}=1400±170 nM); vide infra.

### DETAILED DESCRIPTION OF DRAWINGS

FIG. 1 illustrates the structures of genistein (1) which is the aglycone of genistin (2), daidzein (3) and its corresponding aglycone daidzin (4) and apigenin (5) which was used for comparison with the first two aglycones. The isoflavone daidzein, lacking the hydroxyl group at the 5 position of genistein, is also found in soy foods, but no chemoprotective effects have been attributed to it.

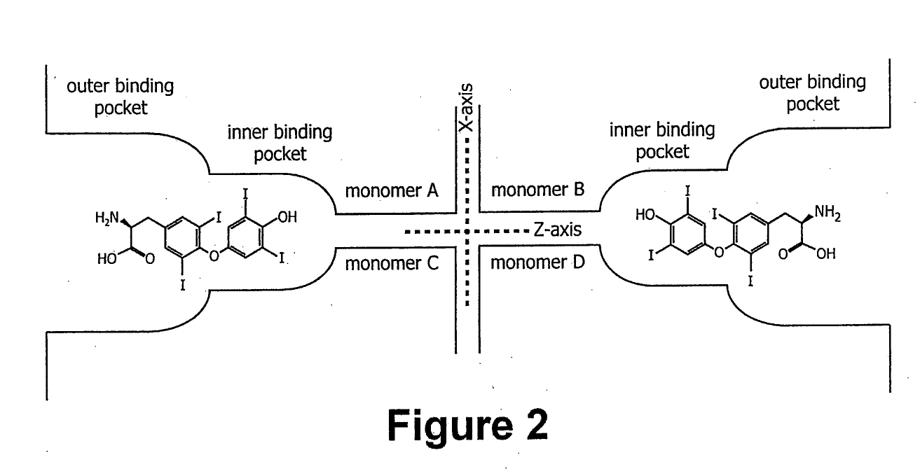

FIG. 2 illustrates a schematic representation of the tetrameric structure of transthyretin depicting the two thyroxine binding sites. The two binding sites are interconverted by two C₂ axes perpendicular to the crystallographic two-fold axes. Each binding site, filled with thyroxine, has an inner and outer binding pocket.

FIG. 3 illustrates a series of three bar graphs comparing the efficacy of the different compounds in preventing fibril formation. Partial acid denaturation-mediated aggregation of (A) WT, (B) V30M, and (C) V122I TTR. Blue bars represent data from an aggregate formation assay wherein tetrameric TTR (3.6 μM) is preincubated with inhibitor (3.6 μM or 7.2 μM) for 30 min prior to lowering the pH to 4.4 (72 h). The Y axis in each bar graph (optical density at 350 nm) represents aggregate formation relative to WT TTR (3.6 μM) assigned as 100%. Hence 5% aggregate formation equates to 95% inhibition.

FIG. 4 illustrates a series of graphs showing the rate of urea-mediated tetramer dissociation (6 M) curves for (A) WT (circles), (B) V30M, and (C) V122I TTR. TTR dissociation is slowed dramatically when WT and the variants are preincubated with genistein (1.8 μM, triangles; 3.6 μM, diamonds). The far-UV CD ellipticity at 214-218 nm was compared to that of WT to determine the fraction of TTR that dissociated and rapidly unfolded at each time point. The lesser effect seen with V30M does not necessarily imply that genistein will be inferior in treating V30M disease, as these experiments employ urea solutions that are unlikely to simulate the physiological conditions in which genistein must be efficacious, rather they are used to demonstrate kinetic stability.

What is claimed is:

1. A method of treating a patient having or potentially having a transthyretin amyloidosis, the method comprising the following step:
administering to said patient a composition containing a therapeutically effective dose of genistein as an active ingredient, said genistein being administered in an amount sufficient for inhibiting acid-mediated fibril-formation of transthyretin in the plasma of said patient by at least about 90 percent during the course of said treatment, said genistein having a structure represented by the formula:

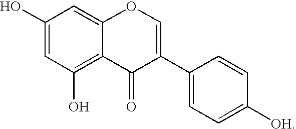

2. The method according to claim 1 wherein the transthyretin amyloidosis is senile systemic amyloidosis.

3. The method according to claim 1 wherein the transthyretin amyloidosis is familial amyloidosis polyneuropathy.

4. The method according to claim 3 wherein the familial amyloidosis polyneuropathy is characterized by V30M mutation.

5. The method according to claim 1 wherein the transthyretin amyloidosis is familial amyloidosis cardiomyopathy.

6. The method according to claim 5 wherein the familial amyloidosis cardiomyopathy is characterized by a V122I mutation.

7. The method according to claim 1 wherein said genistein is administered to said patient in an amount sufficient to raise the plasma concentration of genistein to a level of 3.6 micromolar or greater.

8. The method according to claim 1 wherein said genistein is administered to said patient in an amount sufficient to raise the plasma concentration of genistein to a level of 7.2 micromolar or greater.

9. The method according to claim 1 wherein said administration is repeated periodically.

10. The method according to claim 1 wherein said genistein is administered to the patient orally.

11. The use of genistein in the manufacture of a medicament for the treatment of a patient having or potentially having transthyretin amyloidosis, the medicament containing a therapeutically effective dose of genistein as an active ingredient, said genistein being administered in an amount sufficient for inhibiting acid-mediated fibril-formation of transthyretin in the plasma of said patient by at least about 90 percent during the course of said treatment, said genistein having a structure represented by the formula:

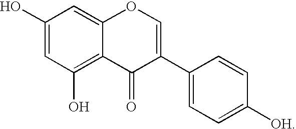

12. The use according to claim 11 wherein the transthyretin amyloidosis is senile systemic amyloidosis.

13. The use according to claim 11 wherein the transthyretin amyloidosis is familial amyloidosis polyneuropathy.

14. The use according to claim 13 wherein the familial amyloidosis polyneuropathy is characterized by V30M mutation.

15. The use according to claim 11 wherein the transthyretin amyloidosis is familial amyloidosis cardiomyopathy.

16. The use according to claim 15 wherein the familial amyloidosis cardiomyopathy is characterized by a V122I mutation.

17. The use according to claim 11 wherein said genistein is administered to said patient in an amount sufficient to raise the plasma concentration of genistein to a level of 3.6 micromolar or greater.

18. The use according to claim 11 wherein said genistein is administered to said patient in an amount sufficient to raise the plasma concentration of genistein to a level of 7.2 micromolar or greater.

19. The use according to claim 11 wherein said administration is repeated periodically.

20. The use according to claim 11 wherein said genistein is administered to the patient orally.
